(19) United States
(12) Patent Application Publication
(10) Pub. No.: US 2004/0175786 A1
(43) Pub. Date: Sep. 9, 2004

(54) Anti-cd3 single-chain antibodies having human cmu3 and cmu4 domains

(76) Inventors: Ingrid Choi, Bad Homburg (DE); Melvyn Little, Neckargemund (DE); Inger Sandlie, Oslo (NO)

Correspondence Address: Steven J Hultquist, Intellectual Property Technology Law, PO Box 14329, Research Triangle Park, NC, 27709, US

(21) Appl. No.: 10/181,305

(22) PCT Filed: Jan. 10, 2001
(86) PCT No.: PCT/DE01/00130

(30) Foreign Application Priority Data
Jan. 14, 2000 (DE) 10001372.4

(51) Int. Cl. C12P 21/02; C07H 21/04; C07K 16/28; C12N 5/06

(52) U.S. Cl. 435/69.1; 435/320.1; 530/388.22; 536/23.53; 435/334

(57) ABSTRACT

The invention relates to a single-chain antibody which is characterized in that (a) it contains a variable domain (scFv) binding specifically to human CD3, and (b) its constant domains are derived from a human IgM molecule and comprise the C_{μ}3 domain and the C_{μ}4 domain but not the C_{μ}1 domain and the C_{μ}2 domain. In a preferred embodiment, the antibody according to the invention contains a human IgG₃ hinge region between the variable and constant domains. The invention also relates to DNA sequences coding for this antibody and to expression vectors containing these DNA sequences and finally to preparations containing the above compounds, preferably for the prevention of acute rejection reactions following organ transplantation.

The present invention relates to a single-chain antibody which is characterized in that (a) it contains a variable domain (scFv) binding specifically to human CD3, and (b) the constant domains are derived from a human IgM molecule and comprise the C_{μ}3 domain and C_{μ}4 domain but not the C_{μ}1 domain and C_{μ}2 domain. In a preferred embodiment, the antibody according to the invention contains a human IgG₃ hinge region between the variable and constant domains. The present invention also relates to DNA sequences coding for this antibody and to expression vectors containing these DNA sequences and finally to medicaments containing the above compounds, preferably for preventing acute rejection reactions following organ transplantation.

OKT3 is a monoclonal murine IgG2a antibody against the ε-chain of the CD3 complex on human lymphocytes, which has been used successfully in hospitals for several years to prevent acute rejection reactions after organ transplantations. The use of OKT3 is often the only possibility of controlling acute rejection reactions, above all in patients showing resistance to conventional immunosuppressive agents (e.g. corticosteroids). The administration of OKT3 induces in vivo a major increase in circulating CD3⁺ cells, since it modulates down TCR. During the first few days of treatment, however, severe side-effects can occur. They comprise e.g. chills and fever and the patients sometimes suffer from nausea, vomiting, diarrhea, dyspnea, wheezing breath and serum meningitis. Many of these side-effects are attributed to the release of cytokines, in particular T-cells. In the case of a relatively long administration, intensive immune response to the constant domain of the murine OKT3 antibody occurs as well.

Thus, the present invention is based on the technical problem of providing anti-CD3 antibodies which when administered, e.g. to avoid tissue rejection reactions, do not show the above described side-effects.

This technical problem is solved by providing the embodiments characterized in the claims.

It was possible to show in the present invention that the above described clinical side-effects can be avoided by using a chimeric OKT3 scFv IgM miniantibody containing the coding sequences for the variable light chains (VL) and variable heavy chains (VH) of a stable OKT3 scF mutant (Kipriyanov et al., Protein Eng. 10(4) (1997), 445) and in place of the murine IgG Fc domains of the OKT3 antibody the C_{μ}3 and C_{μ}4 domains of three different heavy human IgM chains, namely IgM wt, IgM C575S and IgM VAEVD (Sorensen et al., J. Immunol 156(8) (1996), 2858). These three species differ structurally merely as regards the tailpiece (μtp). By means of the present invention it was possible to show that although these differences have a clear influence on polymerization pattern and antibody production, they do not influence substantially the binding to CD3 and the immunosuppressive properties. The single-chain antibodies according to the invention bind selectively to T-cells and inhibit the binding of monoclonal OKT3 antibodies. Functional studies also showed that the single-chain antibodies according to the invention induce neither T-cell proliferation nor the production of IL-2, TNF-α and INF-γ but can be compared as regards CD3 modulation and the inhibition of the immune response with monoclonal OKT3 antibodies. Moreover, it can be assumed that due to their dimensions small as compared to a complete IgM molecule the single-chain antibodies according to the invention have improved tissue penetration, which can result e.g. in the fact that the desired effects occur also with minor doses and/or that these effects are more intensive. In addition, it can also be expected that “clearance” from the circulation occurs more rapidly. The lack of C_{μ}1 domain and C_{μ}2 domains further reduces the number of possible non-specific reactions. Finally, another advantage is to be seen in that single-chain antibodies can be produced more easily in recombinant fashion.

Thus, the present invention relates to a single-chain antibody, characterized in that (a) it contains a variable domain (scFv) binding specifically to human CD3, and (b) the constant domains are derived from a human IgM molecule and comprise the C_{μ}3 domain and C_{μ}4 domain but not the C_{μ}1 domain and C_{μ}2 domain.

Methods of producing an antibody having these properties or the DNA coding for such an antibody, its expression in suitable hosts and its preparation and purification are known to a person skilled in the art and also described in WO 89/09622, WO 89/01783, EP-A10 239 400, WO 90/07861 and Colcher et al., Cancer Research 49 (1989), 1732-1745, for example. The person skilled in the art can also further modify the corresponding immunoglobulin chains, e.g. by deleting, inserting, substituting and/or recombining amino acids. Methods of introducing such modifications into the DNA sequence coding for the immunoglobulin chain are known to the person skilled in the art; see e.g. Sambrook Molecular Cloning—A Laboratory Manual, Cold Spring Harbor Laboratory (1989), N.Y. In order to obtain a domain representing the variable part of the antibody (scFv; shortened Fab fragment which consists of the variable region of a heavy chain and the variable region of a light chain, linked by an artificial peptide member) which binds specifically to human CD3, the person skilled in the art can use previously published sequences as a basis, e.g. the sequence of the murine monoclonal antibody OKT3 according to the method described in below example 1. In order to obtain of a constant domain of the antibody by deleting the DNA coding for the C_{μ}1 and C_{μ}2 domains, the person skilled in the art can also use previously published sequences, e.g. according to the methods described in below Example 1. The person skilled in the art also knows what to observe when he wants to link these DNAs coding for said domains and what additional elements the DNA coding for the single-chain antibody has to contain, e.g. the “tailpiece” (μtp), the “leader” sequence of VH chain and hinge region. Reference is made to Olafsen, T. et al., Immunotech 4 (1998), 141-153 and Norderhang, L. et al., J. Immunol. Method (1997), 77-87, for example.

In a preferred embodiment, the variable domain binding specifically to human CD3 is scFv of the murine monoclonal antibody OKT3.

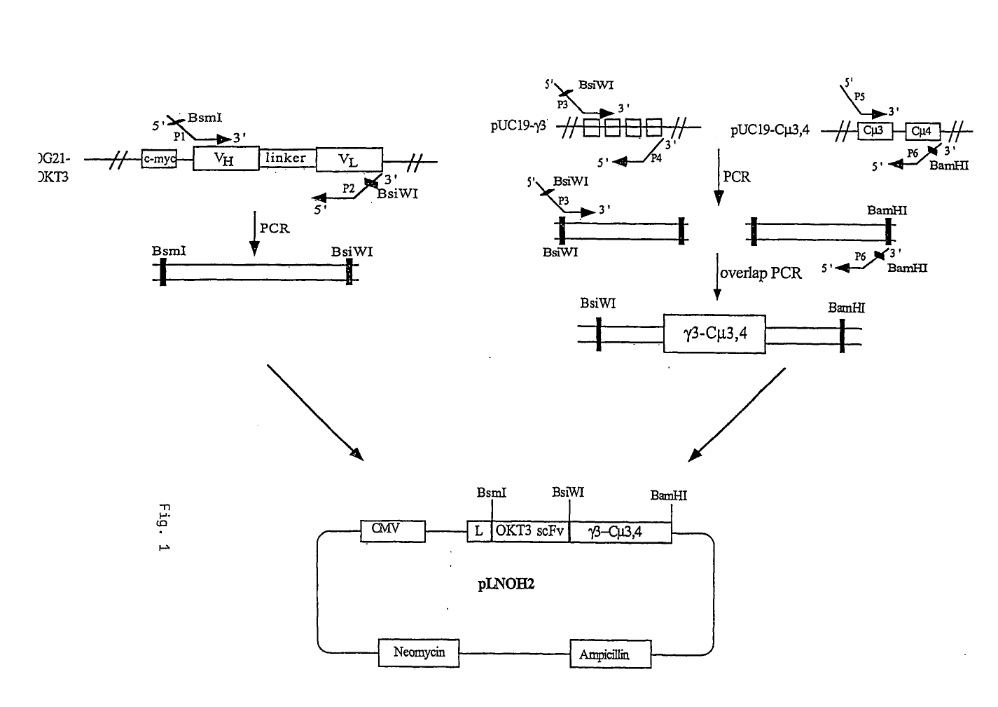

In a particularly preferred embodiment, the single-chain antibody according to the invention has a human IgG₃ hinge region between variable domain and constant domains. As a result, additional stability and mobility is obtained between antigen binding site and constant domain. The introduction of this hinge region can be carried out by methods with which the person skilled in the art is familiar, e.g. as described in below Example 1 and shown in the diagram of FIG. 1.

A more preferred embodiment of the single-chain antibody according to the invention contains a C_{μ}3 domain and a C_{μ}4 domain, derived from the C575 or VAEVD mutant. As compared to the wild-type these mutants differ as regards the tailpiece (μtp). As shown earlier, mutation of cysteine at position 575 in the IgM tailpiece prevents the incorporation of the J chain. No J chain could be identified in the IgM VAEVD mutant either. Thus, it can be assumed that the scOKT3-γΔIgM constructs are even less immunogenic with mutants C575 or VAEVD than the scOKT3-γΔIgM-wt construct.

In an even more preferred embodiment, the single-chain antibody according to the invention is available in polymeric form, e.g. as a dimer, tetramer, pentamer, hexamer or a mixture thereof. These forms distinguish themselves over the monomeric ones by an increased avidity, the pentameric forms additionally resulting in a stronger inhibition of T-cell proliferation. The polymeric forms of the antibody can be obtained by generally known methods, e.g. by means of the fractionation of the culture supernatant, described in Example 1, via gel filtration, e.g. using Superdex-200.

Another preferred embodiment of the present invention relates to DNA sequences coding for the single-chain antibody according to the invention. As to the production of these DNA sequences reference is made to the explanations made above in connection with the single-chain antibody per se.

The DNA sequences according to the invention can also be inserted in a vector or expression vector. Thus, the present invention also comprises vectors and expression vectors containing these DNA sequences. The term “vector” refers to a plasmid (pUC18, pBR322, pBlueScript, etc.), to a virus or another suitable vehicle. In a preferred embodiment, the DNA molecule according to the invention is functionally linked in an expression vector with regulatory elements permitting the expression thereof in prokaryotic or eukaryotic host cells. Along with the regulatory elements, e.g. a promoter, such vectors typically contain a replication origin and specific genes permitting the phenotypic selection of a transformed host cell. The regulatory elements for the expression in prokaryotes, e.g. E. coli, comprise the lac, trp promoter or T7 promoter, and those for the expression in eukaryotes comprise the AOX1 or GAL1 promoter in yeast, and the CMV-, SV40-, RVS-40 promoter, CMV or SV40 enhancer is used for the expression in animal cells. Further examples of suitable promoters are the metallothionein I promoter and the polyhedrin promoter. Suitable expression vectors for E. coli are e.g. pGEMEX, pUC derivatives and pGEX-2T. pY100 and Ycpad1 are counted among the vectors suited for expression in yeast, and pMSXND, pKCR, pEFBOS, cDM8 and pCEV4 are counted among those suited for expression in mammalian cells. The pLNOH2 expression vector is particularly preferred.

General methods known in the art can be used for the construction of expression vectors which contain the DNA sequences according to the invention and suitable control sequences. These methods comprise e.g. in vitro recombination techniques, synthetic methods and in vivo recombination methods, as described in Sambrook et al., supra, for example. The DNA sequences according to the invention can also be inserted in combination with a DNA coding for another protein or peptide, so that the DNA sequences according to the invention can be expressed in the form of a fusion protein, for example.

The present invention also relates to host cells containing the above described vectors. These host cells comprise bacteria (e.g. the E. coli strains HB101, DH1, x1776, JM101, JM109, BL21, and SG13009), yeast, preferably S. cerevisiae, insect cells, preferably sf9 cells, and animal cells, preferably mammalian cells. Preferred mammalian cells are myeloma cells, the murine myeloma cell line Ag8 K2/k being particularly preferred. Methods of transforming these host cells for the phenotypic selection of transformants and for the expression of the DNA molecules according to the invention using the above described vectors are known in the art.

The present invention also relates to methods for the recombinant production of the single-chain antibody according to the invention using the expression vectors according to the invention. The method according to the invention comprises culturing the above described host cells under conditions permitting the expression of the protein (or fusion protein) (preferably stable expression), and obtaining the protein from the culture or from the host cells. The person skilled in the art knows conditions of culturing transformed or transfected host cells. Suitable purification methods (e.g. preparative chromatography, affinity chromatography, immunoaffinity chromatography, e.g. by means of anti-human IgM sepharose, HPLC, etc.) are also generally known.

The present invention permits the implementation of therapeutic measures, i.e. it can be used for preventing acute rejection reactions after organ transplantations, for example. Hence the present invention also relates to a medicament containing the above described single-chain antibodies, DNA sequences or expression vectors according to the invention. Where appropriate, this medicament additionally contains a pharmaceutically compatible carrier. Suitable carriers and the formulation of such medicaments are known to the person skilled in the art. Suitable carriers are e.g. phosphate-buffered common salt solutions, water, emulsions, e.g. oil/water emulsions, wetting agents, sterile solutions, etc. The medicaments are administered orally or parenterally. The methods of parenteral administration comprise the topical, intra-arterial, intra-muscular, subcutaneous, intramedullary, intrathekal, intraventricular, intravenous, intraperitoneal or intranasal administration. The suitable dose is determined by the attending physician and depends on different factors, e.g. the patient's age, sex and weight, the kind of administration, etc.

The above-described DNA sequences are preferably inserted in a vector suited for gene therapy, e.g. under the control of a tissue-specific promoter, and introduced into the cells. In a preferred embodiment, the vector containing the above described DNA sequences is a virus, e.g. an adenovirus, vaccinia virus or an adeno-associated virus. Retroviruses are particularly preferred. Examples of suitable retroviruses are MoMuLV, HaMuSV, MuMTV, RSV or GaLV. For the purpose of gene therapy, the DNA sequences according to the invention can also be transported to the target cells in the form of colloidal dispersions. They comprise e.g. liposomes or lipoplexes (Mannino et al., Biotechniques 6 (1988), 682).

Finally, the present invention relates to the use of the single-chain antibody according to the invention, to the DNA sequence coding for it and to the expression vector containing this DNA sequence for immunosuppression, e.g. for treating colorectal carcinoma, an HIV infection and autoimmune diseases. Its use for immunosuppression serving for preventing acute rejection reactions after organ transplantations is preferred.

The figures show:

FIG. 1: Cloning diagram of the scOKT3-γΔIgM constructs

A stable OKT3 scFv mutant, the γ3 hinge region and the C_{μ}3/C_{μ}4 domain of C_{μ} wild-type, of the C_{μ} C575S and C_{μ} VAEVD variants were amplified by means of PCR from various plasmids, restriction sites having been produced for the purpose of cloning into the expression vectors pLNOH2 which contains gene cassettes for V and C genes.

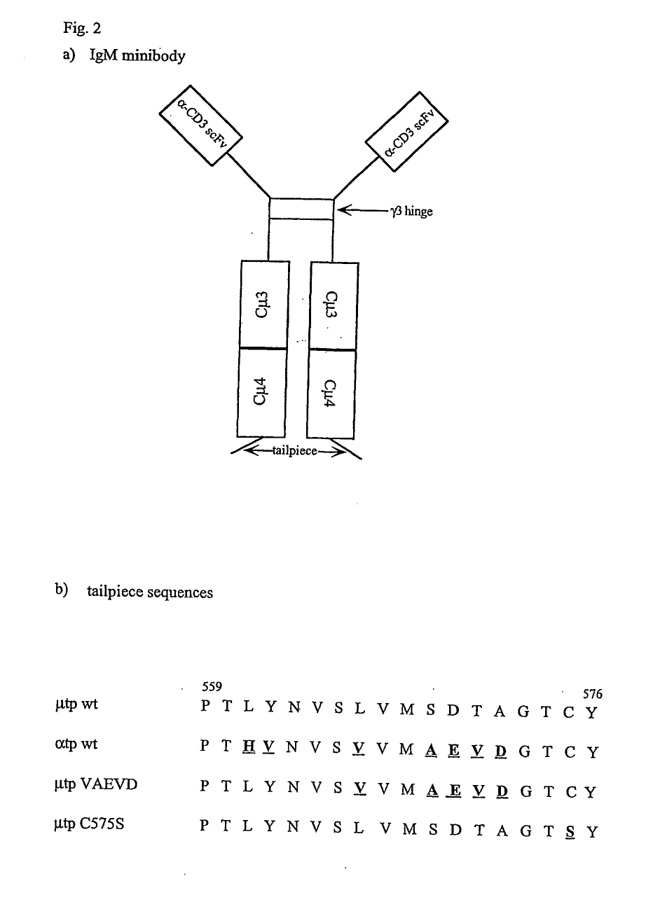

FIG. 2:

(a) Diagram of an OKT3 scFv IgM miniantibody monomer

scOKT3-γΔIgM constructs combine via disulfide bridges to form bivalent structures resembling a typical monomeric IgM molecule.

(b) Amino acid sequences of different tailpieces

IgA wt (αtp wt) (SEQ ID NO: 1) and the three IgM variants (μtp wt (SEQ ID NO: 2), C575S (SEQ ID NO: 3) and VAEVD (SEQ ID NO: 4))

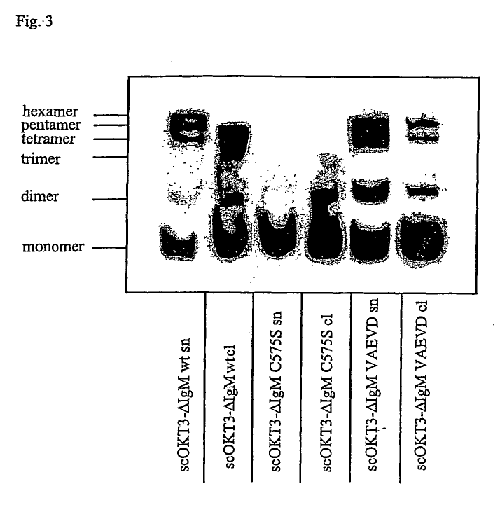

FIG. 3: Polymerization patterns of scOKT3-γΔIgM constructs

Deglycosylated scOKT3-γΔIgM samples from cell lysates (cl) and supernatants (sn) were analyzed using a non-reducing composed 4% SDS acrylamide/agarose gel. Following electroblots on nitrocellulose membranes and incubation with an HRP-conjugated goat-anti-human-IgM detection antibody, the blots were developed by means of chemiluminescence and an autoradiography film was exposed therewith for 3 hours.

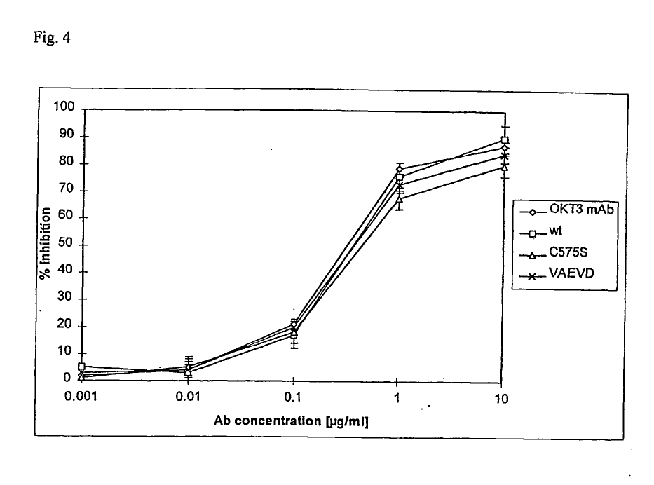

FIG. 4: OKT3 displacement assay

Human CD3⁺ Jurkat cells were incubated at different dilutions of each OKT3 antibody (scOKT3-γΔIgM antibodies and monclonal OKT3 antibodies) for 1 hour. A saturating amount of 10 μg/ml OKT3-PE (OKT3 phycoerythrin) was added. After another hour, the cells were washed and bound OKT3-PE was quantified via FACS analysis. Values are expressed as percent inhibition of the maximum fluorescence intensity, which was determined by adding OKT3-PE in the absence of blocking antibodies. Average values from two experiments were made and the standard deviation was quantified.

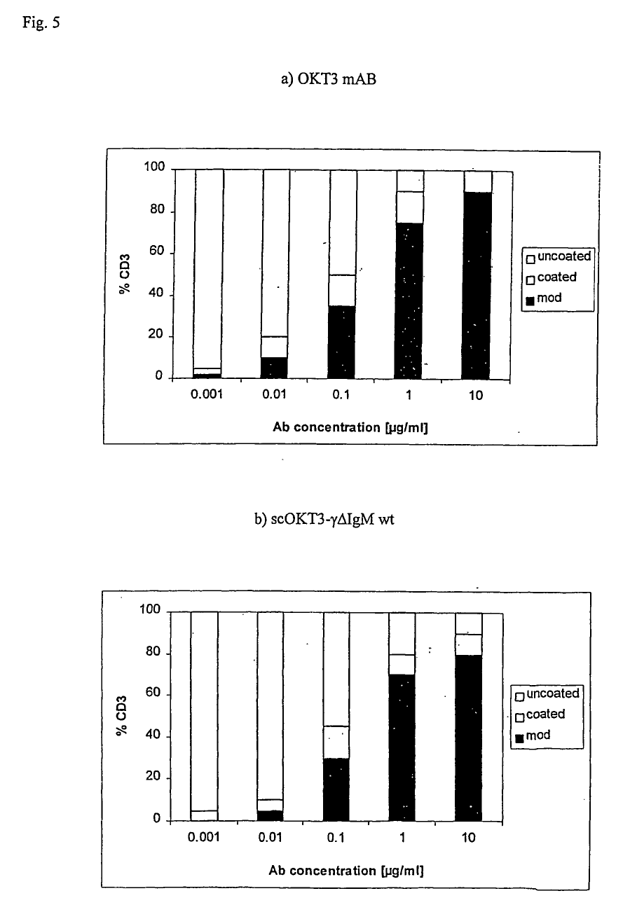

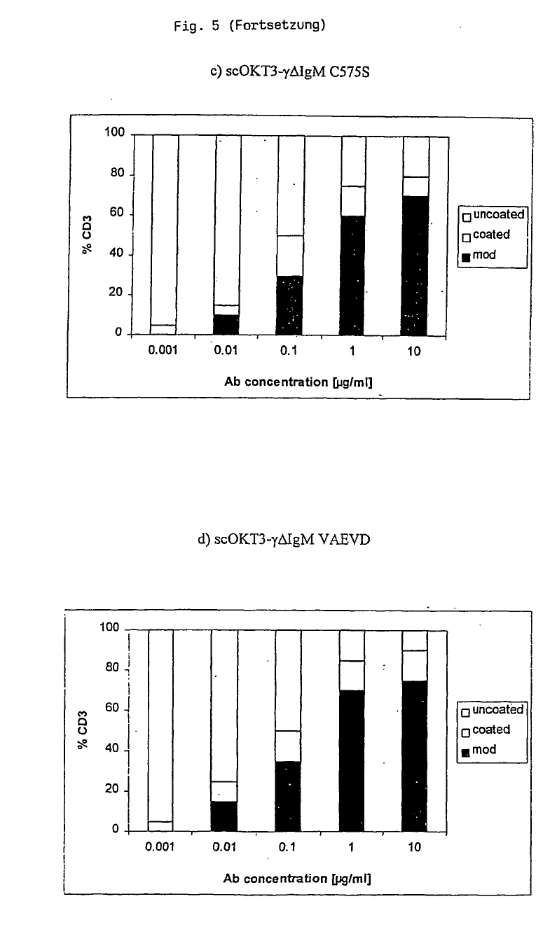

FIG. 5: CD3 modulation and coating

Human mononuclear cells of the peripheral blood (PBMC) were incubated for 12 hours at different dilutions of OKT3 antibodies (scOKT3-γΔIgM antibodies and monoclonal OKT3 antibodies). CD3 modulation and coating were analyzed by means of FACS. The data represent the percentage of surface CD3 on cells which had been treated with OKT3 antibodies, expressed as a fraction of the surface CD3 expressed by control cells.

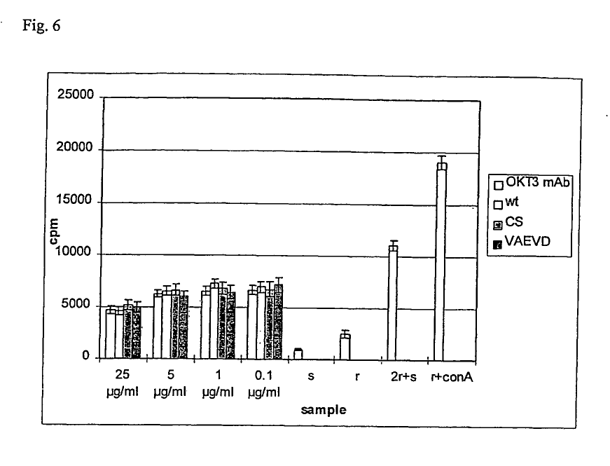

FIG. 6: Inhibition of T-cell proliferation by OKT3 antibodies (scOKT3-γΔIgM antibodies and monoclonal OKT3 antibodies)

HLA B7⁺ “responder”(r) cells were incubated with HLA B7⁻ “stimulator”(s) cells at a ratio of 2:1 at different dilutions of OKT3 antibodies. After 72 hours, the cells were pulse-labeled using [³H] thymidine and the incorporation of radioactivity was measured. Furthermore, “responder” and irradiated “stimulator” cells were incubated both alone and together without OKT3 antibodies. “Responder” cells treated with 5 μg/ml concavalin A were used as a positive control.

The below examples explain the invention.

### EXAMPLE 1

### General Method

(A) DNA constructs: DNA coding for the region of a modified OKT3 scFv (Kipriyanov et al., Protein Eng. 10(4) (1997), 445) was isolated from the plasmid pHOG21-dmOKT3 (Kipriyanov, S. M. et al., Prot. Eng. 10(4), (1997), 445-453) using the PCR primers P1 (SEQ ID NO: 5) (scFv primer 5′-GGTGTGCATTCCCAGGTGCAGCTGCAGCAGTC-3′; the BsmI site is underlined) and P2 (SEQ ID NO: 6) (scFv primer 5′-GACGTACGACTCACCCCGGTTTATTTCCAACTTTGTC-3′; the BsiWI site is underlined), by means of which restriction sites BsiWI and BsmI were introduced. This fragment was then cloned into the vector pLNOH2 cleaved using BsiWI/BsmI (Norderhaug et al., J. Immunol. Methods 204(1) (1997), 77) in VH-VL orientation. For adding the γ₃—C_{μ}3C_{μ}4 region, the IgG3 hinge wild-type gene contained in the pUC19 Cγ₃ plasmid (Olafsen, T. et al., Cancer Immunol. Immunother. 48, (1999), 411-418) was first amplified with the PCR primers P3 (SEQ ID NO: 7) (“hinge” primer 5′-GGCCAGCGTACGGAGGGAGGGTGTCT-3′; the BsWI site is underlined) and P4 (SEQ ID NO: 8) (“hinge” primer 5′-GTGTTCTTGATCTGAGGAAGAGATGGAGGCAGATG-3′). The restriction site, BsWI site, necessary for cloning was produced by site-directed mutagenesis using the P3 primer. Thereafter, the matrixes consisting of pUC19 C_{μ} wt, pUC19 C_{μ} VAEVD and pSV2 C_{μ} C575S (human IgM C_{μ}3 and C_{μ}4 domains of WT and the VAEVD and C575S mutations) Störensen, V. et al., J. Immunol. 156, (1996), 2853-2865) were used for amplifying the C_{μ}3 and C_{μ}4 regions. The PCR reactions were carried out with the primers P5 (SEQ ID NO: 9) (C_{μ}3,4-primer 5′-CATCTCTTCCTCAGATCAAGACACAGCCATCCG-3′) and P6 (SEQ ID NO: 10) (C_{μ}3,4-primer 5′-ACTCAGGATCCGTATCTTTTGAATGG-3′; the BamHI site is underlined), a BamHI restriction site having been introduced at the 3′ end of each gene for the C_{μ}variants. In the subsequent step, the γ₃ and C_{μ} fragments were amplified using PCR splicing by overlapping extension reactions with the primers P3 and P6. Each γ₃-tIgM construct was then cloned separately into the pLNOH2-αCD3 scFv expression vector cleaved using BsiWI/BamHI (FIG. 1). The three resulting gene constructs scOKT3-γΔIgM (OKT3 scFv-γ₃ “hinge”-reduced IgM) were verified by sequencing.

(B) Cell cultures and transfections: All of the cell lines were cultured at 37° C. in a damp atmosphere containing 5% CO₂. The cells were kept in RPMI 1640 supplemented with 10% fetal calf serum (FCS), 100 U/ml penicillin, 100 μg/ml streptomycin and 2 mM L-glutamine. The three scOKT3-γΔIgM constructs were introduced separately into the murine myeloma cells Ag8 K2/k (Deutsches Krebsforschungszentrum, Heidelberg, Germany) by electroporation. After 72 hours, selective medium was added up to a final concentration of 500 μg/ml G418. Three weeks later, the resistant transfectants were screened by means of ELISA using an anti-human IgM-HRP detection antibody as regards the secretion of recombinant protein. Positive transfectants were subcloned by boundary dilution methods. The three best-produced scOKT3-γΔIgM clones of each variant were then expanded.

(C) Purification of scOKT3-γΔIgM proteins: The expressed proteins were purified by means of anti-human IgM sepharose. The column was washed with application buffer (50 mM Na phosphate, pH 7.0) until the OD_{280 nm} of flow-through was below 0.01. scOKT3-γΔIgM antibody was eluted using 0.1 M acetic acid. The collected fractions were immediately neutralized with 1.0 M Tris-HCl, pH 9.0, and dialyzed against PBS.

(D) Cell lyses, deglycosylation and Western blot analyses: 1×10⁶ cells of each selected clone were used for the Western blots. The supernatants were collected after 24 hours and the cells were washed two times with ice-cold PBS. They were then incubated in 50 mM Tris-HCl, pH 7.5, 150 mM NaCl, 1% NP-40 (supplemented with a protease inhibitor up to a final concentration of 4 mM) at 4° C. for 30 min. The recombinant antibodies of the cell lyzates and supernatants were purified as described above and deglycosylated with PNGase F under non-reducing conditions for 4 hours. Samples of the cell lyzates and supernatants were analyzed by means of 10% SDS-PAGE under reducing conditions. In order to determine the polymer formation, the samples were also separated under non-reducing conditions on 4% SDS acrylamide/agarose mixed gel as described recently (Sorensen et al., J. Immunol. 156(8) (1996), 2858). Both gels were electroblotted on nitrocellulose membranes. The membranes were blocked with 2% milk powder from skimmed milk/PBS and incubated with HRP-conjugated goat-anti-human IgM (dilution 1:2000). After thorough washing, the blots were developed by means of chemiluminescence and an exposition of an autoradiography film was carried out.

(E) Isolation of polymer fractions: The polymer mixture of IgM mutant-containing culture supernatants was separated by means of gel filtration using Superdex-200 in various fractions.

(F) “Displacement” assay: 1×10⁶ CD3⁺ Jurkat cells (human T-cell lymphoma line) in PBS were washed per sample and incubated at different concentrations of the respective scOKT3-γΔIgM antibodies and monoclonal OKT3 antibodies at 4° C. for 1 hour. Following washing, a saturating amount (10 μg/ml) of OKT3-PE was added and the cells were incubated at 4° C. for 1 hour and then washed. The intensity of fluorescence was determined by means of FACS analysis. Human CD3-JOK-1 cells (human B-cell lymphoma line) were used as a negative control. scOKT3-γΔIgM polymer fractions were analyzed by means of FACS analysis using the same method.

(G) Proliferation assay: Human mononuclear cells of the peripheral blood (PBMC) were isolated by means of density gradient centrifugation on a “Ficoll-Hypaque” gradient (Sigman) from the blood of a healthy donor (26 years old, female). PBMC were resuspended in modified Iscov medium supplemented with an autologous serum and aliquoted in microtitration plate wells having a round bottom (plates having 96 wells each) (4×10⁵ cells/well). The induction of T-cell proliferation was analyzed using a saturation concentration of soluble and plastic-immobilized OKT3 antibodies (scOKT3-γΔIgM antibodies and monoclonal OKT3 antibodies). PBMC were incubated together with the OKT3 antibodies for 72 hours. Then, the cells were pulse-labeled with 1 μCi [³H]thymidine/well. 18 hours later, the cells were collected and the incorporation of radioactivity was determined by means of a liquid scintillation IS counter. The activation capability of the polymer fractions was also determined. Native IgM was used as a control. T-cell activation was carried out in the presence of the costimulating substrates IL-2 (25 U/ml) and monoclonal anti-CD28 antibodies (10 μg/well).

(H) Determining IL-2, TNF-α and INF-γ: PBMC were plated out and the antibodies were immobilized as described for T-cell proliferation. The induction of IL-2, TNF-α and INF-γproductions was determined with a saturation concentration of the OKT3 antibodies (scOKT3-γΔIgM antibodies and monoclonal OKT3 antibodies). Supernatants were collected after 24 hours (to determine IL-2), 36 hours (TNF-α) and 72 hours (INF-γ). Commercially available ELISA kits were used to quantify the concentrations of the cytokines secreted into the medium. Native IgM was used as a control.

(I) Quantification of CD3 coating and modulation: PBMC were incubated at a concentration of 1×10⁶ cells/ml for 12 hours in 24-well plates at various concentrations of the OKT3 antibodies (scOKT3-γΔIgM antibodies and monoclonal OKT3 antibodies). PBMC of each group were collected and stained with the following substances: (1) goat-anti-mouse FITC (fluoresceinisothiocyanate) (anti-Mig-FITC), (2) 10 μg/ml OKT3 for 30 min. and then anti-Mig-FITC, or (3) OKT3-FITC. Each FITC-conjugated antibody was used at a dilution of 1:100. For identifying the T-lymphocytes, the fluorescein-stained cells were counterstained using anti-human CD5-PE (dilution 1:100) and analyzed by means of FACS. The calculations for CD3 coating and modulation were carried out using the following formula described by Woodle et al. (Transplantation 52(2) (1991), 354):

$$\begin{matrix} fraction CD3 \text{-} coated = \frac{mAk \text{-} treated cells {MC}_{anti \text{-} MIg \text{-} FITC} - control cells {MC}_{anti \text{-} MIg \text{-} FITC}}{control cells {MC}_{10 µg OKT3 / anti \text{-} MIg \text{-} FITC} - control cells {MC}_{anti \text{-} MIg \text{-} FITC}} & ( 1 ) \\ \% CD3 coating = 100 \% \times fraction CD3 coating & ( 2 ) \\ {CD3}_{non \text{-} modulated} fraction = \frac{mAk \text{-} treated cells {MC}_{10 µg OKT3 / anti \text{-} MIg \text{-} FITC} - control cells {MC}_{anti \text{-} MIg \text{-} FITC}}{control cells {MC}_{10 µg OKT3 / anti \text{-} MIg \text{-} FITC} - control cells {MC}_{anti \text{-} MIg \text{-} FITC}} & ( 3 )\end{matrix}$$

% modulated CD3=100%−(fraction CD3 non-modulated×100) (4)

MC represents the middle channel along the x-axis. Isolated polymer fractions were tested separately.

(J) Mixed lymphocyte culture (MLC): For determining the immunosuppressive potential of the scOKT3-γΔIgM antibodies and monoclonal OKT3 antibodies as regards the inhibition of T-cell proliferation, PBMC from two healthy donors (“responder”: 30 years old, male, HLA B7⁺; “stimulator”: 31 years old, male, HLA B7⁻) were isolated and kept as described for the proliferation assay. The “stimulator” cells were γ-irradiated with 30 Gy and plated out into 96-well microtitration plates at a density of 10⁵ cells/well. Then 2×10⁵ “responder” cells were added to each well. After 72 hours of incubation at different dilutions of OKT3 antibodies, the cells were pulse-labeled with 3 μCi [³H]-thymidine/well. The cells were collected 12 hours later and the incorporation of radioactivity was determined in a liquid scintillation β counter. In order to determine background proliferation, irradiated “stimulator” cells and “responder” cells were analyzed separately. T-cell proliferation of “responder” cells treated with concavalin A at a final concentration of 5 μg/ml was measured as a positive control.

### EXAMPLE 2

### Expression and Purification of scOKT3-γΔIgM

For the production of recombinant IgM miniantibodies of OKT3, a gene was constructed as in Example 1, which codes for a leader sequence derived from a HV gene of anti-NIP hybridoma, a stable OKT3 scFv mutant, a human γ3 hinge exon and exons of human IgM C_{μ}3 and C_{μ}4 Fc domains (Wt, C575S and VAEVD mutants). The gene construct has a size of about 3.0 kbp. The correctness of the sequence was determined by means of sequence analysis after ligation into the expression vector pLNOH2 which contains restriction sites for cassette cloning of any intact V region followed by any C region. An IgM miniantibody is shown by way of diagram in FIG. 2.

The expression constructs were transfected into the myeloma cells Ag8 K2/k. For detecting an antibody expression, the clones were screened following selection via ELISA. Stably transfected Ag8 K2/k clones produced about 1-2 μg/ml scOKT3-γΔIgM with the exception of the wild-type clones whose antibody secretion were only about 1 tenth (Table 1).

TABLE 1
Antibody production of stable Ag8 K2/k transfectants

| Antibodies | antibody productions |
|---|---|
| scOKT3-γΔIgM, WT | 0.1 (+/−0.08) |
| scOKT3-γΔIgM, C575S | 2.3 (+/−0.8) |
| scOKT3-γΔIgM, VAEVD | 1.1 (+/−0.6) |

The cell culture supernatants were collected after 24 hours. scOKT3-γΔIgM antibodies were isolated as described in Example 1. The antibody concentration was determined via OD (OD at 280 nm corresponds to 0.7 mg/ml, according to the “DNA Gene Inspector” Software). The average values from 3 experiments are given and the standard deviations are shown in parentheses.

A study of the cell lyzates showed that the low production of scOKT3-γΔIgM-WT antibodies was not based on a greater intracellular restraint of the non-degraded protein. The scOKT3-γΔIgM-C575S mutant secreted about twice as many antibodies as compared to the scOKT3-γΔIgM-VAEVD mutant. The three best-produced clones of each IgM variant were expanded.

The culture supernatants and cell lyzates of the selected Ag8 K2/k clones were analyzed by immunoblots under reducing conditions with and without glycosides using an HRP(horseradish peroxidase)-conjugated anti-human IgM antibody. Deglycosilated scOKT3-γΔIgM constructs showed a band at the expected size of 60 kDa, whereas the mobility of the glycosilated products corresponded to a size of about 67 kDa. Under non-reducing conditions, the manipulated IgM constructs occurred as a mixture of monomers and polymers (FIG. 3). scOKT3-γΔIgM-WT secreted polymers comprising hexamers, pentamers and tetramers, whereas the scOKT3-γΔIgM-VAEVD mutant screted polymers which were predominantly intermediates such as pentamers, tetramers and dimers.

On the contrary, the scOKT3-γΔIgM-C575S construct only secreted monomers into the supernatant. The cell lyzates showed a somewhat different antibody polymerization pattern. No hexamers and more intermediate polymers were found in the scOKT3-γΔIgM-WT lyzate. The cell lyzate of the scOKT3-γΔIgM-VAEVD mutant contained a greater amount of monomers as compared to the supernatant. Both monomers and dimers were found in the scOKT3-γΔIgM-C575S lyzate. In order to measure the relative amounts of secreted polymeric forms of scOKT3-γΔIgM antibodies, the supernatants were separated on a superdex-200 gel filtration column and the concentrations of the protein fractions were analyzed by means of OD. The results are shown in Table 2. The monomer contamination in the polymer fractions was below 5%. It was not possible to separate pentamers and hexamers from each other and they were eluted as one fraction.

TABLE 2
Polymerization ratio of the scOKT3-γΔIgM constructs

| | scOKT3- | scOKT3- | scOKT3- |
|---|---|---|---|
| | γΔIgM, | γΔIgM, | γΔIgM, |
| | WT | C575S | VAEVD |
| 24.6% (+/−3.4% | 95.5% (+/−2.6%) | 27.4% (+/−2.8%) | dimers, |
| | | | 240 kDa |
| 6.1% (+/−0.7%) | n.d. | 18.2% (+/−2.5%) | trimers, |
| | | | 360 kDa |
| 2.7% (+/−0.8%) | n.d. | 5.4% (+/−1.2%) | tetramers, |
| | | | 480 kDa |
| 16.4% (+/−1.2%) | n.d. | 23.7% (+/−0.9%) | pentamers/- |
| | | | hexamers |
| | | | 600 kDa/ |
| | | | 720 kDa |
| 44.3% (+/−3.7) | n.d. | 20.5% (+/−2.1%) | |

The results from three experiments were averaged and standard deviations are indicated in parentheses. The first column gives the approximate molecular weights.
n.d.: not determined.

### EXAMPLE 3

### Specificity and Affinity of scOKT3-γΔIgM

As an introductory step to determine the functional integrity of the scOKT3-γΔIgM antibodies their capability of inhibiting the binding of OKT3-PE to the TCR/CD3 complex was checked (FIG. 4). The efficiency of inhibiting OKT3-PE binding to T-cells by the monoclonal OKT3-antibodies and the OKT3 miniantibodies was quantified in a “displacement” assay. The results show that the scOKT3-γΔIgM constructs do not only bind to T-cells but also inhibit competitively the binding of the monoclonal OKT3 antibodies. Furthermore, these studies showed that scOKT3-γΔIgM antibodies have binding affinities which resemble those of the parental murine antibodies. In order to be able to detect in the polymer fractions of the scOKT3-γΔIgM antibodies differences as regards the capability of binding, they were incubated separately with Jurkat cells. As expected, antibodies with higher polymerization degree showed greater affinity to CD3 than the monomers. Almost 80% of the Jurkat cells showed positive staining with scOKT3-γΔIgM monomers and over 90% of the Jurkat cells showed this positive staining with scOKT3-γΔIgM pentamers and the monoclonal OKT3 antibodies.

### EXAMPLE 4

### T-cell Activation by scOKT3-γΔIgM

T-cell proliferation as a response to monoclonal OKT3 antibodies and scOKT3-γΔIgM was tested with human PBMC. For determining the influence of the TCR/CD3 cross-linkage on T-cell proliferation, assays were made with soluble and immobilized OKT3 antibodies (Table 3). Soluble scOKT3-γΔIgM antibodies induced minimum proliferation. Contrary to immobilized monoclonal OKT3 antibodies, plastic-immobilized scOKT3-γΔIgM antibodies only showed little T-cell activation. No difference was detected after stimulation with monomers or higher polymers. This indicates that multivalent TCR/CD3 cross-linkage induced no proliferation.

TABLE 3
Induction of T-cell activation by anti-CD3 antibodies
(monoclonal OKT3 antibodies and
scOKT3-γΔIgM antibodies)

| | | immobilized Ak |
|---|---|---|
| | Soluble Ak [cpm × 10³] | (cpm × 10³] |
| OKT3 mAk | 9.5 (+/−1.5) | 12.5 (+/−1.9) |
| scOKT3-γΔIgM, WT | 2.6 (+/−0.5) | 4.6 (+/−0.8) |
| scOKT3-γΔIgM, C575S | 1.9 (+/−0.3) | 3.3 (+/−0.7) |
| scOKT3-γΔIgM, VAEVD | 2.3 (+/−0.8) | 3.7 (+/−1.1) |
| scOKT3-γΔIgM, WT | 1.8 (+/−0.4) | 4.0 (+/−0.7) |
| Monomers | | |
| scOKT3-γΔIgM, WT | 2.1 (+/−0.4) | 3.5 (+/−0.4) |
| Pentamers/hexamers | | |
| scOKT3-γΔIgM, C575S | 2.4 (+/−0.5) | 3.7 (+/−0.9) |
| Monomers | | |
| scOKT3-γΔIgM, VAEVD | 2.9 (+/−1.1) | 4.1 (+/−0.2) |
| Monomers | | |
| scOKT3-γΔIgM, VAEVD | 3.2 (+/−0.7) | 4.4 (+/−1.0) |
| Pentamers | | |

The data +/− standard deviation were corrected by subtraction of cpm from PBMC cultures without antibodies. The results from three experiments were averaged and standard deviations are given in parentheses.

T-cell co-stimulation with human IL-2 and monoclonal anti-CD28 antibodies resulted in an increase in the proliferation with all the anti-CD3 antibodies. This confirms that the secreted scOKT3-γΔIgM constructs bind to TCR/CD3 without intensive activation.

The influence of the antibody concentration on the production of IL-2, TNF-α and INF-γ was determined by means of ELISA. Human BPMC were incubated at a saturation concentration of 10 μg/ml with each anti-CD3 antibody for 24 hours (IL-2), 36 hours (TNF-α) or 72 hours (INF-γ) and the tissue supernatants were collected. As compared to their parental counterpart, the scOKT3-γΔIgM antibodies induced no significant cytokine release. When scOKT3-γΔIgM monomers or polymers were used, only slight differences were observed as regards the production of IL-2, TNF-α and INF-γ (Table 4). At the time of collection of the supernatant, PBMC cultured with monoclonal OKT3 antibodies and scOKT3-γΔIgM antibodies were studied as regards the viability by means of the “trypan blue exclusion” test. It showed that the plurality of cells was viable after each induction.

TABLE 4
Induction of cytokine release by OKT3 antibodies
(OKT3-mAk and scOKT3-γΔIgM)

| | | TNF-α- | INF-γ- |
|---|---|---|---|
| | IL-2 production | production | production |
| | [pg/ml] | [pg/ml] | [pg/ml] |
| OKT3 mAk | 467 (+/−129) | 960 (+/−157) | 1381 (+/89) |
| scOKT3-γΔIgM, | 47 (/+/−32) | 167 (+/23) | 244 (+/−78) |
| WT | | | |
| scOKT3-γΔIgM, | 65 (+/−26) | 149 (+/43) | 202 (+/95) |
| C575S | | | |
| scOKT3-γΔIgM, | 79 (+/−12) | 157 (+/−47) | 194 (+/−28) |
| VAEVD | | | |
| scOKT3-γΔIgM, | 52 (+/37) | 166 (+/−13) | 222 (+/−56) |
| WT | | | |
| Monomers | | | |
| scOKT3-IgM, | 42 (+/−22) | 158 (+/−24) | 232 (+/−47) |
| WT | | | |
| Pentamers/ | | | |
| Hexamers | | | |
| scOKT3-γΔIgM, | 73 (+/−31) | 169 (+/−38) | 189 (+/−67) |
| C575S, monomers | | | |
| scOKT3-γΔIgM, | 67 (+/−11) | 143 (+/−69) | 224 (+/−28) |
| VAEVD, monomers | | | |
| scOKT3-γΔIgM, | 72 (+/−17) | 154 (+/−65) | 198 (+/−49) |
| VAEVD, | | | |
| pentamers | | | |

The data +/− standard deviation were corrected by subtracting cpm from PBMC cultures without antibodies. The results from three experiments were averaged and standard deviations are given in parentheses.

### EXAMPLE 5

### CD3 Coating and Modification

In order to be able to study the immunosuppressive potential of the scOKT3-γΔIgM antibodies as compared to that of OKT3-mAk, the coating and modulation of CD3 were quantified (FIG. 5). Although the entire amount of CD3 coated or modulated by the IgM miniantibodies was similar to that observed with mAk, maximum CD3 modulation induced by the miniantibodies was somewhat less than that induced by mAk with each studied concentration. The slightest effect on CD3 modulation was observed when the scOKT3-γΔIgM constructs were used which had only been produced as monomers. Besides that no significant differences as regards the degree of CD3 modulation induced by scOKT3-γΔIgM pentamers or mAk was found. This indicates that the recombinant constructs with higher valence are as effective as their monoclonal counterparts as regards CD3 modulation.

### EXAMPLE 6

### Immunosuppression

The immunosuppressive properties of different Ak were studied in vitro by studying their capacity as regards the suppression of an immune response induced in MLC. The scOKT3-γΔIgM antibodies inhibited T-cell proliferation efficiently at concentrations corresponding to those achieved with OKT3-mAk (FIG. 6). This effect was slightly increased when the scOKT3-γΔIgM pentamer fractions were used.

1. A single-chain antibody, characterized in that
a) it contains a variable domain (scFv) binding specifically to human CD3, and
b) its constant domains are derived from a human IgM molecule and comprise the C_{μ}3 domain and the C_{μ}4 domain but not the C_{μ}1 domain and C_{μ}2 domain.

2. The single-chain antibody according to claim 1, wherein the variable domain binding specifically to human CD3 is the scFv of murine monoclonal OKT3.

3. The single-chain antibody according to claim 1 or 2, which has a human IgG₃ hinge region between the variable domain and the constant domains.

4. The single-chain antibody according to any one of claims 1 to 3, wherein the C_{μ}3 domain and C_{μ}4 domain are derived from the C575S or VAEVD mutant.

5. The single-chain antibody according to any one of claims 1 to 5, which is present in polymeric form.

6. A DNA sequence, encoding the single-chain antibody according to any one of claims 1 to 5.

7. An expression vector, containing the DNA sequence according to claim 6 which is functionally linked with a promoter.

8. The expression vector according to claim 7, which is pLNOH2.

9. A cell line, containing the expression vector according to claim 7 or 8.

10. The cell line according to claim 9, which is a murine myeloma cell line.

11. The cell line according to claim 10, which is Ag8 K2/k.

12. A pharmaceutical preparation containing the single-chain antibody according to any one of claims 1 to 5, the DNA sequence according to claim 6 or the expression vector according to claim 7 or 8.

13. Use of the antibody defined in the above claims, the DNA sequence or the expression vector for immunosuppression.

14. Use according to claim 13, wherein the immunosuppression relates to the prevention of acute rejection reactions following organ transplantation.

15. A method of preparing the antibody according to any one of claims 1 to 5, characterized in that
(a) a cell line according to any one of claims 9 to 11 is transfected with the expression vector according to claim 7 or 8 and cultured under suitable conditions, and
(b) the expressed protein is obtained from the cell line or the culture and purified.


SEQUENCE LISTING (10 sequences)

SEQ ID NO: 1 - protein, 18 aa, Artificial Sequence
  Synthetic Construct
PTLYNVSLVMSDTAGTCY

SEQ ID NO: 2 - protein, 18 aa, Artificial Sequence
  Synthetic Construct
PTHVNVSVVMAQVDGTCY

SEQ ID NO: 3 - protein, 18 aa, Artificial Sequence
  Synthetic Construct
PTLYNVSVVMAEVDGTCY

SEQ ID NO: 4 - protein, 18 aa, Artificial Sequence
  Synthetic Construct
PTLYNVSLVMSDTAGTSY

SEQ ID NO: 5 - DNA, 32 bp, Artificial Sequence
  Synthetic Construct
ggtgtgcattcccaggtgcagctgcagcagtc

SEQ ID NO: 6 - DNA, 37 bp, Artificial Sequence
  Synthetic Construct
gacgtacgactcaccccggtttatttccaactttgtc

SEQ ID NO: 7 - DNA, 26 bp, Artificial Sequence
  Synthetic Construct
ggccagcgtacggagggagggtgtct

SEQ ID NO: 8 - DNA, 35 bp, Artificial Sequence
  Synthetic Construct
gtgttcttgatctgaggaagagatggaggcagatg

SEQ ID NO: 9 - DNA, 33 bp, Artificial Sequence
  Synthetic Construct
catctcttcctcagatcaagacacagccatccg

SEQ ID NO: 10 - DNA, 26 bp, Artificial Sequence
  Synthetic Construct
actcaggatccgtatcttttgaatgg